(19) United States
(12) Patent Application Publication
(10) Pub. No.: US 2019/0090411 A1
(43) Pub. Date: Mar. 28, 2019

(54) CYANOBACTERIUM TREATED DICOT PLANT SEEDS AND RELATED METHODS

(71) Applicant: Terra Biologics LLC, St. Louis, MO (US)

(72) Inventors: Michael Ott, Iowa City, IA (US); Lawrence E. Page, St. Louis, MO (US)

(21) Appl. No.: 16/139,314

(22) Filed: Sep. 24, 2018

Related U.S. Application Data
(60) Provisional application No. 62/563,056, filed on Sep. 25, 2017.

(51) Int. Cl. A01C 1/06; A01N 63/04; A01N 25/04; C12N 1/14

(52) U.S. Cl. CPC A01C 1/06; C12N 1/14; A01N 25/04; A01N 63/04

(57) ABSTRACT

Dicot seeds treated with various cyanobacterium compositions, methods of making the treated seeds, and methods of using the seeds to improve dicot plant growth, biomass, nutrient uptake, nitrogen uptake, lateral root growth, lateral root hair growth, vertical root growth, root branching and/or yield are provided.

### CROSS REFERENCE TO RELATED APPLICATIONS

This application claims the benefit of U.S. Provisional Patent Application Ser. No. 62/563,056 filed on Sep. 25, 2017, the disclosure of which is incorporated herein by reference in its entirety.

### STATEMENT REGARDING FEDERALLY SPONSORED RESEARCH OR DEVELOPMENT

Not applicable

### THE NAMES OF PARTIES TO A JOINT RESEARCH AGREEMENT

Not applicable

### REFERENCE TO A SEQUENCE LISTING, TABLE, OR COMPUTER PROGRAM LISTING APPENDIX SUBMITTED ON A COMPACT DISC AND AN INCORPORATION-BY-REFERENCE OF THE MATERIAL ON A COMPACT DISC

The instant application contains a Sequence Listing as a text file, which is entitled “54886-154378-SEQ-LIST-ST25.txt,” as created on Apr. 5, 2016, and is 2,940 bytes in size. This sequence listing was submitted via EFS-Web in ASCII format on Sep. 25, 2017, and is hereby incorporated by reference in its entirety.

### STATEMENT REGARDING PRIOR DISCLOSURES BY THE INVENTOR OR JOINT INVENTORS

The following disclosure(s) are submitted under 35 U.S.C. § 102(b): WO 2016/164813 published Oct. 13, 2016 to inventors Michael Ott and Lawrence E. Page, and is assigned to Terra Biologics LLC.

### FIELD OF THE INVENTION

Compositions are provided and can be used, for example, in coating seeds or other particles or in methods for improved yield, nitrogen uptake, nutrient uptake, lateral root growth, lateral root hair growth, vertical root growth, root branching and biomass, optionally under nitrogen limiting growth conditions.

### BACKGROUND OF THE INVENTION

Cyanobacteria are photosynthetic prokaryotes of the phylum Cyanobacteria. Although Cyanobacteria have been referred to as “blue-green algae,” it is now recognized that Cyanobacteria should not be considered “algae” as they are prokaryotes rather than eukaryotes.

Treatment of various dicot plants with certain Cyanobacteria to improve dicot plant growth has been reported. Treatment of soil with a Nostoc strain soon after seed germination has been reported to improve maize growth and nitrogen uptake (Maqubela et al., Plant Soil Plant and Soil 315: 79-92, 2009). However, Maqubela et al. also report that the “high rate of application cannot be recommended for field scale application as the amount of inoculum required would be unrealistically high” (Maqubela et al. at page 91). Treatment of rice paddy fields with a mixture of Anabaena, Nostoc, Aulosira and Tolypothrix to improve rice yield has also been reported (Mishra and Pabbi, Resonance 9(6): 6-10, 20040). Although improved methods of producing the cyanobacterial inoculum are reported by Mishra and Pabbi, they report that “the effects of inoculation” are inconsistent and that the best results were obtained when the inocula are produced from local stocks and used with a low level of nitrogenous fertilizer (Mishra and Pabbi at page 9).

Field trials performed using various Cyanobacteria treatments on rice have shown that certain treatments can increase yield by 22% and total nitrogen uptake by 59% (International Patent Application Publication WO 2016/164819). Yield improvements over controls for the Cyanobacteria treatments were most pronounced when the rice was grown without added fertilizer.

Greenhouse experiments performed using various Cyanobacteria treatments on cotton have also shown that certain Cyanobacteria treatments performed under certain soil conditions improved biomass production in comparison to untreated controls when no nitrogen fertilizer is provided (International Patent Application Publication WO 2016/164813).

### BRIEF SUMMARY OF THE INVENTION

A treated dicot plant seed is provided, the seed comprising a solid coating on at least a portion of an outer surface of the seed. The coating comprises cyanobacteria comprised of an Aulosira species and a Tolypothrix species, wherein either:

(1) the coating is free of an agriculturally acceptable adjuvant and/or an agriculturally acceptable excipient; or

(2) the coating further comprises the adjuvant and/or the excipient in an amount less than 0.09 gram per gram of seed; or

(3) yield resulting from the treated dicot plant seed is at least 2.0% greater than yield resulting from the same dicot plant seed that is not treated with the coating when grown under the following greenhouse conditions:

(a) seeds are planted in Captina silt loam soil with application of 40 lb/acre phosphate fertilizer and 20 lb/acre potassium fertilizer with 3 plants per pot for hybrid plant and 6 plants per pot for pureline plant;

(b) temperature maintained at 25-28° C., 50-70% relative humidity and natural light supplemented with fluorescent light at 35 μmol m⁻² s⁻² photosynthetically active radiation to provide a 14 h photoperiod; and

(c) plant grown to 50% heading then entire above-ground biomass is collected and dried, and total weight and nitrogen concentration can be measured; or

(4) the cyanobacteria consists essentially of the Aulosira species and the Tolypothrix species; or

(5) the coating consists essentially of the Aulosira species and the Tolypothrix species; or

(6) the coating comprises the Aulosira species and the Tolypothrix species in a weight ratio of about 5:1 to about 1:5.

Also provided is a method of obtaining a plurality of dicot crop plants with improved yield, nitrogen uptake, nutrient uptake, biomass, lateral root growth, lateral root hair growth, vertical root growth or root branching, the method comprising: distributing the treated dicot plant seed on a plot of land and cultivating plants grown from the seed on the plot.

A method of obtaining a dicot plant seed that provides for improved growth in a dicot plant obtained from the seed is also provided. The method comprises applying a composition comprising the cyanobacteria to the at least a portion of an outer surface of the seed and lyophilizing or drying the composition to form the solid coating and obtain the treated dicot plant seed.

Also provided is a method of improving yield, nitrogen uptake, nutrient uptake, biomass, lateral root growth, lateral root hair growth, vertical root growth or root branching in a dicot plant comprising:

exposing the dicot plant seed to an effective amount of a composition comprising cyanobacteria comprised of an Aulosira species and a Tolypothrix species, wherein either:

(1) the composition is free of an agriculturally acceptable adjuvant and/or an agriculturally acceptable excipient; or

(2) the composition further comprises the adjuvant and/or the excipient in an amount less than 0.4 kg/hectare; or

(3) the cyanobacteria consists essentially of the Aulosira species and the Tolypothrix species; or

(4) the composition consists essentially of the Aulosira species and the Tolypothrix species; or

(5) the composition comprises the Aulosira species and the Tolypothrix species in a weight ratio of about 5:1 to about 1:5; and

allowing the dicot plant seed to germinate;

wherein either:

(A) yield resulting from the treated dicot plant seed is at least 2.0% greater than yield resulting from the same dicot plant seed that is not exposed to the composition when grown under greenhouse conditions; or

(B) nutrient uptake resulting from the treated dicot plant seed is at least 2% greater than nitrogen uptake resulting from the same dicot plant seed that is not exposed to the composition when grown under the greenhouse conditions;

(C) nitrogen uptake resulting from the treated dicot plant seed is at least 3% greater than nitrogen uptake resulting from the same dicot plant seed that is not exposed to the composition when grown under the greenhouse conditions; or

(D) biomass resulting from the treated dicot plant seed is at least 2% greater than biomass resulting from the same dicot plant seed that is not exposed to the composition when grown under the greenhouse conditions; or

(E) lateral root growth, lateral root hair growth, vertical root growth or root branching resulting from the treated dicot plant seed exhibits increased branching as compared to lateral root growth, lateral root hair growth, vertical root growth or root branching resulting from the same dicot plant seed that is not exposed to the composition when grown under the greenhouse conditions;

wherein the greenhouse conditions are as follows:

(a) seeds are planted in Captina silt loam soil with application of 40 lb/acre phosphate fertilizer and 20 lb/acre potassium fertilizer with 3 plants per pot for hybrid plant and 6 plants per pot for pureline plant;

(b) temperature maintained at 25-28° C., 50-70% relative humidity and natural light supplemented with fluorescent light at 35 μmol m⁻² s⁻² photosynthetically active radiation to provide a 14 h photoperiod; and

(c) plant grown to 50% heading then entire above-ground biomass is collected and dried, and total weight and nitrogen concentration can be measured.

Other objects and features will be in part apparent and in part pointed out hereinafter.

### BRIEF DESCRIPTION OF THE SEVERAL VIEWS OF THE DRAWING

FIG. 1 shows weight and weight measurements from the cotton greenhouse trial. The listed figures are an average of the two soil types, Crevasses and Loring. Addition of the cyanobacteria showed increases in height of 15.45% and weight of 24.73%, compared to an untreated control.

FIG. 2 shows differences in roots observed in the greenhouse trial. Roots from untreated control seeds had less root biomass, branching and depth than roots from seeds that had been treated with cyanobacterial mixture.

### DETAILED DESCRIPTION OF THE INVENTION

It has been discovered that seeds can be coated with the minimal amount of cyanobacteria which can provide a commercially significant increase in yield, nitrogen uptake, nutrient uptake, biomass, lateral root growth, lateral root hair growth, vertical root growth and/or root branching. The seed coating need not contain any ingredients other than cyanobacteria if desired so that the coating can be comprised of entirely biologically active ingredients. The cyanobacteria is sufficiently tacky to adhere to the seed without the need for other tackifiers or other typical coating ingredients.

Provided herein are dicot plant seeds treated with various cyanobacterial compositions that provide improved dicot plant growth characteristics, methods of making the treated seeds, and methods of using the seeds. Such improved growth characteristics conferred by the seed treatments include, but are not limited to, improved nitrogen uptake, increased nutrient uptake, increased lateral root growth, increased lateral root hair growth, increased vertical root growth, increased root branching, and increased yield under nitrogen limiting growth conditions. Also provided are dicot plant seeds, methods of making, and methods of use where the seeds are treated with combinations of cyanobacterium species that can effect more pronounced improvements in the aforementioned growth characteristics when compared to untreated seeds or seeds treated with other combinations of cyanobacterium species.

A treated dicot plant seed is provided, the seed comprising a solid coating on at least a portion of an outer surface of the seed. The coating comprises cyanobacteria comprised of an Aulosira species and a Tolypothrix species, wherein either:

(1) the coating is free of an agriculturally acceptable adjuvant and/or an agriculturally acceptable excipient; or

(2) the coating further comprises the adjuvant and/or the excipient in an amount less than 0.09 gram per gram of seed; or

(3) yield resulting from the treated dicot plant seed is at least 2.0% greater than yield resulting from the same dicot plant seed that is not treated with the coating when grown under the following greenhouse conditions:

(a) seeds are planted in Captina silt loam soil with application of 40 lb/acre phosphate fertilizer and 20 lb/acre potassium fertilizer with 3 plants per pot for hybrid plant and 6 plants per pot for pureline plant;

(b) temperature maintained at 25-28° C., 50-70% relative humidity and natural light supplemented with fluorescent light at 35 μmol m⁻² s⁻² photosynthetically active radiation to provide a 14 h photoperiod; and

(c) plant grown to 50% heading then entire above-ground biomass is collected and dried, and total weight and nitrogen concentration can be measured; or

(4) the cyanobacteria consists essentially of the Aulosira species and the Tolypothrix species; or

(5) the coating consists essentially of the Aulosira species and the Tolypothrix species; or

(6) the coating comprises the Aulosira species and the Tolypothrix species in a weight ratio of about 5:1 to about 1:5.

The coating can be free of an agriculturally acceptable adjuvant or an agriculturally acceptable excipient, or can be free of both the adjuvant and excipient.

If an amount of the adjuvant or excipient is present in the coating, the coating comprises the adjuvant and/or the excipient in an amount of at least 0.001 gram per gram of seed. The adjuvant or excipient can be in an amount less than 0.08, 0.07, 0.06, 0.05, 0.04, 0.03, 0.02 or 0.01 gram per gram of seed.

The cyanobacteria can consist essentially of the Aulosira species and the Tolypothrix species.

The coating can consist essentially of the Aulosira species and the Tolypothrix species.

Also provided herein are treated dicot plant seeds that have been at least partially coated with a composition comprising: (i) at least one cyanobacterium species; and (ii) an agriculturally acceptable adjuvant, an agriculturally acceptable excipient, or a combination thereof.

Compositions used to treat dicot plant seeds can comprise (i) at least one cyanobacterium species; and (ii) an agriculturally acceptable adjuvant, an agriculturally acceptable excipient, or a combination thereof.

Cyanobacteria used in the compositions and coatings provided herein include, but are not limited to, cyanobacteria in the order Nostocales.

The composition or coating can comprise one, two, three, or four cyanobacterium species that are members of the family Nostocaceae and/or at least one cyanobacterium species that is a member of the family Microchaetaceae.

The member or members of the family Nostocaceae can comprise a Nostoc sp., an Aulosira sp., and/or an Anabaena sp., and the member of the family Microchaetaceae can comprise a Tolypothrix sp.

Non-limiting examples of a Nostoc sp., an Aulosira sp., an Anabaena sp., and a Tolypothrix sp. and cultures comprising the same that can be used in the compositions or coatings are provided in Table 1.

TABLE 1
Cyanobacterium species

| | | | Geographic Origin |
|---|---|---|---|
| | | | of Source |
| Genus | Species | Source¹ | Organism |
| Nostoc | commune | Nostoc commune | Austin, Texas; USA |
| | | UTEX B 1621 | |
| Aulosira | bohemensis | Aulosira bohemensis | Dlouha Ves, South |
| | | UTEX B 2947 | Bohemia; Czech |
| | | | Republic |
| Anabaena | cylindrica | Anabaena cylindrica | England |
| | | UTEX B 1611 | |
| Tolypothrix | distorta | Tolypothrix distorta | Utrecht, Utrecht; |
| | | UTEX 424 | Netherlands |

¹All strains with a “UTEX” designation are publicly available through the University of Texas at Austin UTEX Culture Collection of Algae, 1 University Station A6700, Austin, TX, USA or via the internet on the world wide web site “utex.org.”

The member or members of the family Nostocaceae can comprise a Nostoc commune UTEX B 1621 culture, an Aulosira bohemensis UTEX B 2947 culture, an Anabaena cylindrica UTEX B 1611 culture, isolate(s) therefrom, or a variant thereof and the member or members of the family Microchaetaceae can comprise a Tolypothrix distorta UTEX 424 culture, isolate(s) therefrom, or a variant thereof.

Cyanobacterium that can be used in the compositions, coatings and methods described herein can also be identified by the sequence of the gene encoding the 16S RNA. The cyanobacteria that are used can be characterized by having a gene encoding a 16S RNA that has at least about 95%, 97%, 98%, 99%, 99.5%, 99.7%, 99.9%, or 100% sequence identity across the entire length of SEQ ID NO:1, SEQ ID NO:2, SEQ ID NO:3, and/or SEQ ID NO:4. For example, the cyanobacterium species can be characterized by having a gene encoding a 16S RNA that has at least 95%, 97%, 98%, 99%, 99.5%, 99.7%, 99.9%, or 100% sequence identity across the entire length of SEQ ID NO:1 and/or SEQ ID NO:4.

Useful 16S RNA sequences are provided in Table 2.

TABLE 2
Cyanobacterium 16S RNA sequences

| | SEQ | |
|---|---|---|
| | ID | Consensus Sequence of |
| Genus | NO: | gene encoding 16S RNA1 |
| Aulosira | 1 | CGAAAGCCTGACGGAGCAATACCGCGTGAGGGAGG |
| | | AAGGCTCTTGGGTCGTAAACCTCTTTTCTCAGGGA |
| | | AGAACAAAATGACGGTACCTGAGGAATAAGCATCG |
| | | GCTAACTCCGTGCCAGCAGCCGCGGTAATACGGAG |
| | | GATGCAAGCGTTATCCGGAATGATTGGGCGTAAGC |
| | | GTCCGCAGGTGGCTATGTAAGTCTGCTGTTAAAGA |
| | | GTGAGGCTCAACCTCATAAAAGCAGTGGAAACTAC |
| | | ATGGCTAGAGTGCGTTCGGGGCAGAGGGAATTCCT |
| | | GGTGTAGCGGTGAAATGCGTAGAGATCAGGAAGAA |
| | | CACCGGTGGCGAAAGCGCTCTGCTAGGCCGCAACT |
| | | GACACTGAGGGACGAAAGCTAGGGGAGCGAATGGG |
| | | ATTAGATACCCCAGTAGTCCTGAGACTCCAAGGCA |
| | | cacaGGGGATA |
| Anabaena | 2 | CGAAAGCCTGACGGAGCAATACCGCGTGAGGGAGG |
| | | AAGGCTCTTGGGTCGTAAACCTCTTTTCTCAGGGA |
| | | AGAAaaaTGACGGTACCTGAGGAATAAGCATCGGC |
| | | TAACTCCGTGCCAGCAGCCGCGGTAATACGGAGGA |
| | | TGCAAGCGTTATCCGGAATGATTGGGCGTAAGCGT |
| | | CCGCAGGTGGCCATGTAAGTCTGCTGTTAAAGAGT |
| | | CATGCTTAACATGATAAAAGCAGTGGAAACTACAG |
| | | AGCTAGAGTACGTTCGGGGCAGAGGGAATTCCTGG |
| | | TGTAGCGGTGAAATGCGTAGATATCAGGAAGAACA |
| | | CCGGTGGCGAAAGCGCTCTGCTAGGCCGTAACTGA |
| | | CACTGAGGGACGAAAGCTAGGGGAGCGAATGGGAT |
| | | TAGATACCCCAGTAGT |
| Nostoc | 3 | CGAAAGCCTGACGGAGCAATACCGCGTGAGGGAGG |
| | | AAGGCTCTTGGGT |
| | | TGTAAACCTCTTTTCTCAGGGAATAAAATGAAGGT |
| | | ACCTGAGGAATCAG |
| | | CATCGGCTAACTCCGTGCCAGCAGCCGCGGTAATA |
| | | CGGAGGATGCAAGC |
| | | GTTATCCGGAATGATTGGGCGTAAGCGTCCGCAGG |
| | | TGGCGATGTAAGTC |
| | | TGCTGTTAAAGAGTGAGGCTTAAACCTCATAAAGC |
| | | AGTGGAAACTACAT |
| | | CGCTAGAGTGCGTTCGGGGCAGAGGGAATTCCTGG |
| | | TGTAGCGGTGAAAT |
| | | GCGTAGAGATCAGGAAGAACACCGGTGGCGAAGGC |
| | | GCTCTGCTAGGCCG |
| | | CAACTGACACTGAGGGACGAAAGCTAGGGGAGCGA |
| | | A |
| Tolypothrix | 4 | CGAAAGCCTGACGGAGCAATACCGCGTGAGGGAGG |
| | | AAGGCTCTTGGGTTG |
| | | TAAACCTCTTTTCTCAGGGAAGAATTAAATGACGG |
| | | TACCTGAGGAATAAGC |
| | | ATCGGCTAACTCCGTGCCAGCAGCCGCGGTAATAC |
| | | GGAGGATGCAAGCGTT |
| | | ATCCGGAATGATTGGGCGTAAAGCGTCCGCAGGTG |
| | | GCTATGTAAGTCTGCT |
| | | GTTAAAGAATCTGGCTCAACCAGATAAAGGCAGTG |
| | | GAAACTACATGGCTAG |
| | | AGTGCGTTCGGGGCAGAGGGGAATTTCCTGGTGTA |
| | | GCGGTGAAATGCGTAG |
| | | AGATCAGGAGAACACCGGTGCGAAAGCGCTCTGCT |
| | | AGGCCGCAACTGACAC |
| | | TGAGGGACGAAAGCTAGGGGAGCGAATGGGATTAG |
| | | ATACCCCAGTAGT |

¹16S consensus sequences were generated by harvesting liquid cyanobacterial cultures, amplifying the 16S ribosomal region by PCR, gel-purifying the 16S band, and sequencing the resulting PCR product. Sequences are made up of many reads that were combined to generate a consensus sequence. Lower-case letters in the published sequences denote locations where variation was found in the base at that position such that it reduced confidence to below the set threshold.

The member or members of the family Nostocaceae can be characterized by having a gene encoding a 16S RNA that has at least 95%, 97%, 98%, 99%, 99.5%, 99.7%, 99.9%, or 100% sequence identity across the entire length of SEQ ID NO:1, SEQ ID NO:2, or SEQ ID NO:3, and the member or members of the family Microchaetaceae can be characterized by having a gene encoding a 16S RNA that has at least 95%, 97%, 98%, 99%, 99.5%, 99.7%, 99.9%, or 100% sequence identity across the entire length of SEQ ID NO:4.

The member or members of the family Nostocaceae can be characterized by having a gene encoding a 16S RNA that has at least 95%, 97%, 98%, 99%, 99.5%, 99.7%, 99.9%, or 100% sequence identity across the entire length of SEQ ID NO:1 and the member or members of the family Microchaetaceae can be characterized by having a gene encoding a 16S RNA that has at least 95%, 97%, 98%, 99%, 99.5%, 99.7%, 99.9%, or 100% sequence identity across the entire length of SEQ ID NO:4.

The cyanobacteria can be characterized by having a gene encoding a 16S RNA that has at least 95%, 97%, 98%, 99%, 99.5%, 99.7%, 99.9%, or 100% sequence identity across the entire length of: (i) SEQ ID NO:1; (ii) SEQ ID NO:1 and SEQ ID NO:2; (iii) SEQ ID NO:1 and SEQ ID NO:4; (iv) SEQ ID NO:3 and SEQ ID NO:4; (v) SEQ ID NO:1, SEQ ID NO:2, and SEQ ID NO:3; or (vi) SEQ ID NO:4. For example, cyanobacteria can be characterized by having a gene encoding a 16S RNA that has at least 95%, 97%, 98%, 99%, 99.5%, 99.7%, 99.9%, or 100% sequence identity across the entire length of: (i) SEQ ID NO:1; (ii) SEQ ID NO:1 and SEQ ID NO:2; (iii) SEQ ID NO:1 and SEQ ID NO:4; or (iv) SEQ ID NO:3 and SEQ ID NO:4, or (i) SEQ ID NO:1; (ii) SEQ ID NO:1 and SEQ ID NO:2; (iii) SEQ ID NO:1 and SEQ ID NO:4; (iv) SEQ ID NO:1, SEQ ID NO:2, and SEQ ID NO:3; or (v) SEQ ID NO:4.

The cyanobacteria can comprise any one of: (i) Aulosira sp. and Tolypothrix sp.; (ii) Aulosira sp. and Anabaena sp.; (iii) Aulosira sp.; (iv) Aulosira sp.; Anabaena sp.; and Tolypothrix sp.; or (v) Nostoc sp.

The cyanobacteria can comprise any one of: (i) Aulosira bohemensis sp.; and a Tolypothrix distorta; (ii) Aulosira bohemensis; (iii) Aulosira bohemensis and Anabaena cylindrica; (iv) Aulosira bohemensis, Anabaena cylindrical, and Tolypothrix distorta; or (v) Nostoc commune.

The coating or composition comprises the cyanobacterium in an amount from about 0.001 g to about 0.15 gram per gram of seed, such as in an amount less than 0.08, 0.09, 0.10, 0.11, 0.12, 0.13, or 0.14 gram per gram of seed.

A cyanobacterium useful in compositions, coatings and methods provided herein can also be obtained by isolation from terrestrial sources including, but not limited to soil and plants, as well as aquatic sources.

One useful method for isolating cyanobacteria that can be used involves nutrient saturated glass fiber filters in combination with a broad spectrum beta-lactam antibiotic to isolate cyanobacteria (Ferris and Hirsch, Appl. Environ. Microbio, 57(5): 1448-1452, 1991). Additional methods for obtaining cyanobacteria that use phototaxis and scored agar surfaces in the presence of certain antibiotics can also be used (Vaara et al. Appl. Environ. Microbiol. 38(5):1011-4, 1979). Isolation methods including by not limited to those described by the aforementioned citations and references cited therein can also be used to obtain isolates from non-axenic cyanobacteria cultures to obtain cyanobacterium isolates that can be used in the methods, coatings and compositions provided herein. The isolates are obtained from a non-axenic Nostoc commune UTEX B 1621 culture, a non-axenic Aulosira bohemensis UTEX B 2947 culture, a non-axenic Anabaena cylindrica UTEX B 1611 culture, and a non-axenic Tolypothrix distorta UTEX 424 culture.

Variants of the cyanobacteria can also be used in compositions, coatings and methods provided herein. The cyanobacteria variants are variants that have been induced by mutagenesis and selected for one or more useful traits. Mutagenesis techniques include, but are not limited to use of alkylating agents, intercalating agents, transposons, and the like. Cyanobacteria variants can be screened and then selected for useful traits that include, but are not limited to, increased phototaxis, changes in genome copy number, increased protein production, or altered pigment expression. Variants can also be obtained where the cyanobacteria have been genetically transformed with a heterologous transgene. Methods for transforming cyanobacteria that have been disclosed (Chaurasia, et al., J Microbiol Methods. 73(2):133-41, 2008) can be used to obtain such variants.

Seeds treated with compositions or coatings provided herein can be used to improve a variety of dicot plant growth characteristics including, but not limited to, plant biomass, yield, lateral root growth, lateral root hair growth, vertical root growth, root branching, growth under nitrogen limiting conditions, nutrient uptake and/or nitrogen uptake as discussed in more detail below. The improvement in the dicot plant growth characteristic can be in comparison to the growth characteristic of a dicot plant either obtained from an untreated dicot plant seed or a dicot plant that has been treated with a topical application of cyanobacteria. For example, the improvement in the dicot plant growth characteristic can be in comparison to the growth characteristic of either a dicot plant that has been treated with a topical application of a cyanobacterial mixture or a dicot plant obtained from a dicot plant seed treated with a cyanobacterial mixture, the cyanobacterial mixture comprising a Nostoc commune UTEX B 1621 culture, a Aulosira bohemensis UTEX B 2947 culture, a Anabaena cylindrica UTEX B 1611 culture, and a Tolypothrix distorta UTEX 424 culture.

Dicot seeds can be treated with a composition or coating comprising: (i) Aulosira sp., Anabaena sp., and Tolypothrix sp., or (ii) Nostoc sp.

Dicot seeds can be treated with a composition or coating comprising Aulosira sp., Anabaena sp., Tolypothrix sp., and Nostoc sp.

The Aulosira sp., Anabaena sp., Nostocsp., and/or Tolypothrix sp. used in the composition or coating can be characterized by having at least about 95%, 97%, 98%, 99%, 99.5%, 99.7%, 99.9%, or 100% sequence identity across the entire length of SEQ ID NO:1, SEQ ID NO:2, SEQ ID NO:3, and/or SEQ ID NO:4. For example, the Aulosira sp., Anabaena sp., Nostoc sp., and/or Tolypothrix sp. used in the composition or coating can be characterized by having at least about 95%, 97%, 98%, 99%, 99.5%, 99.7%, 99.9%, or 100% sequence identity across the entire length of SEQ ID NO:1, SEQ ID NO:2, SEQ ID NO:3, or SEQ ID NO:4.

The Aulosira species can be characterized by having a gene encoding a 16S RNA that has at least 95%, 97%, 98%, 99%, 99.5%, 99.7%, 99.9%, or 100% sequence identity across the entire length of SEQ ID NO:1 and the Tolypothrix species are characterized by having a gene encoding a 16S RNA that has at least 95%, 97%, 98%, 99%, 99.5%, 99.7%, 99.9%, or 100% sequence identity across the entire length of SEQ ID NO:4.

The Aulosira species can be characterized by having a gene encoding a 16S RNA that has at least 95%, 97%, 98%, 99%, 99.5%, 99.7%, 99.9%, or 100% sequence identity across the entire length of SEQ ID NO:1.

The Tolypothrix species can be characterized by having a gene encoding a 16S RNA that has at least 95%, 97%, 98%, 99%, 99.5%, 99.7%, 99.9%, or 100% sequence identity across the entire length of SEQ ID NO:4.

The Aulosira species can be an Aulosira bohemensis UTEX B 2947 culture or variant thereof.

The Tolypothrix species can be a Tolypothrix distorta UTEX 424 culture or a variant thereof.

Both an Aulosira bohemensis UTEX B 2947 culture or variant thereof and a Tolypothrix distorta UTEX 424 culture or variant thereof can be used in the methods and seed treatments described herein.

The treated dicot plant seeds can be at least partially coated with a composition comprising: an Aulosira species and a Tolypothrix species at a ratio of 3:1 to 1.5:1, respectively; and (ii) an agriculturally acceptable adjuvant, an agriculturally acceptable excipient, or a combination thereof.

Dicot seeds can be treated with a composition or coating comprising: (i) an Aulosira bohemensis sp. and a Tolypothrix distorta sp.; (ii) an Aulosira bohemensis sp.; (iii) an Aulosira bohemensis sp. and an Anabaena cylindrica sp.; (iv) an Aulosira bohemensis sp., an Anabaena cylindrica sp., and a Tolypothrix distorta sp.; or (v) a Nostoc commune sp.

It may be desirable that (i) the cyanobacterium is not associated with the plant seed in nature; (ii) the composition or coating comprises at least two cyanobacterium species that are not associated with the plant seed in nature; (iii) the composition or coating comprises at least two cyanobacterium species that are not associated with one another in nature; or (iv) the composition or coating comprises an exogeneous exopolysaccharide (EPS). For example, it may be desirable that the Aulosira species and the Tolypothrix species are not associated with the dicot plant seed in nature.

The Aulosira species and the Tolypothrix species may not be associated with the plant seed in nature, or the composition or coating may comprise an exogenous exopolysaccharide (EPS).

It may be desirable that the composition or coating does not further comprise an Anabaena species, a Nostoc species, or both an Anabaena species and a Nostoc species.

It may be desirable that the composition or coating does not further comprise Tolypothrix tenuis, Aulosira fertilissima, and a Nostoc species, or Tolypothrix tenuis, Aulosira fertilissima, a Nostoc species, and a Cylindrospermum species.

Representative examples of cyanobacteria that can be used in the compositions, coatings and methods provided herein that are characterized by having at least about 95%, 97%, 98%, 99%, 99.5%, 99.7%, 99.9%, or 100% sequence identity across the entire length of SEQ ID NO:1, SEQ ID NO:2, SEQ ID NO:3, and/or SEQ ID NO:4 include, but are not limited to, one or more of the cyanobacteria provided in Table 3. Representative examples of cyanobacteria that can be used in the compositions, coatings and methods provided herein also include, but are not limited to, combinations of the cyanobacteria provided in Tables 1 and 3.

The cyanobacterium used to treat the seed can be heterologous to the dicot seed. The cyanobacterium are heterologous to the seed when they are not detectable in or on a seed that has not been treated with the compositions or coatings provided herein.

TABLE 3
Representative Cyanobacteria having 16S RNA Sequences with significant
sequence identity to SEQ ID NO: 1, 2, 3, and/or 4

| | | | | |
|---|---|---|---|---|
| | 16S sequence | | | |
| | % identity to | | | Culture |
| | specified SEQ | NCBI | Culture | Collection |
| Cyanobacterium | ID NO: | Accession¹ | Collection² | Location |

Isolates having a 16S RNA encoding gene with at least
95% sequence identity to Aulosira bohemensis 16S SEQ ID NO: 1

| | | | | |
|---|---|---|---|---|
| Calothrix sp. | 99% | NC_019682.1 | Pasteur | Paris, |
| PCC 7507 | | | Culture | France |
| | | | Collection | |
| Anabaena | 97% | NC_019771.1 | Pasteur | Paris, |
| cylindrica PCC | | | Culture | France |
| 7122 | | | Collection | |
| Cylindrospermum | 97% | NC_019757.1 | Pasteur | Paris, |
| stagnale PCC | | | Culture | France |
| 7417 | | | Collection | |
| Nostoc | 97% | NC_010628.1 | Pasteur | Paris, |
| punctiforme PCC | | | Culture | France |
| 73102 | | | Collection | |
| Nostoc sp. PCC | 96% | NC_003272.1 | Pasteur | Paris, |
| 7120 | | | Culture | France |
| | | | Collection | |
| Nodularia | 96% | NZ_CP007203.1 | Culture | Bratislava, |
| spumigena | | | collection of | Slovakia |
| CCY9414 | | | yeasts | |
| Nostoc azollae | 96% | NC_014248.1 | None | N/A |
| 0708 | | | | |
| Anabaena | 96% | NC_007413.1 | American | Manassas, |
| variabilis ATCC | | | Type Culture | Virginia, |
| 29413 | | | Collection | USA |
| Nodularia | 96% | CP007203.2 | Culture | Bratislava, |
| spumigena | | | collection of | Slovakia |
| CCY9414 | | | yeasts | |
| Nostoc sp. PCC | 95% | NC_019676.1 | Pasteur | Paris, |
| 7107 | | | Culture | France |
| | | | Collection | |
| Anabaena sp. 90 | 95% | NC_019427.1 | University of | Helsinki, |
| | | | Helisinki | Finland |
| | | | Cyanobacterial | |
| | | | Culture | |
| | | | Collection | |

Isolates having at least 95% sequence identity to
Anabaena cylindrica 16S SEQ ID NO: 2

| | | | | |
|---|---|---|---|---|
| Anabaena | 97% | NC_019771.1 | Pasteur | Paris, |
| cylindrica PCC | | | Culture | France |
| 7122 | | | Collection | |
| Calothrix sp. | 99% | NC_019682.1 | Pasteur | Paris, |
| PCC 7507 | | | Culture | France |
| | | | Collection | |
| Nostoc | 97% | NC_010628.1 | Pasteur | Paris, |
| punctiforme PCC | | | Culture | France |
| 73102 | | | Collection | |
| Nostoc sp. PCC | 96% | NC_003272.1 | Pasteur | Paris, |
| 7120 | | | Culture | France |
| | | | Collection | |
| Cylindrospermum | 97% | NC_019757.1 | Pasteur | Paris, |
| stagnale PCC | | | Culture | France |
| 7417 | | | Collection | |
| Nostoc sp. PCC | 95% | NC_019676.1 | Pasteur | Paris, |
| 7107 | | | Culture | France |
| | | | Collection | |
| Anabaena | 96% | NC_007413.1 | American | Manassas, |
| variabilis ATCC | | | Type Culture | Virginia, |
| 29413 | | | Collection | USA |
| Nodularia | 96% | NZ_CP007203.1 | Culture | Bratislava, |
| spumigena | | | collection of | Slovakia |
| CCY9414 | | | yeasts | |
| Anabaena sp. 90 | 95% | NC_019427.1 | University of | Helsinki, |
| | | | Helsinki | Finland |
| | | | Cyanobacterial | |
| | | | Culture | |
| | | | Collection | |
| Nostoc azollae | 96% | NC_014248.1 | None | N/A |
| 0708 | | | | |
| Nodularia | 96% | CP007203.2 | Culture | Bratislava, |
| spumigena | | | collection of | Slovakia |
| CCY9414 | | | yeasts | |

Isolates having at least 95% sequence identity to
Nostoc commune 16S SEQ ID NO: 3

| | | | | |
|---|---|---|---|---|
| Cylindrospermum | 97% | NC_019757.1 | Pasteur | Paris, |
| stagnale PCC | | | Culture | France |
| 7417 | | | Collection | |
| Calothrix sp. | 99% | NC_019682.1 | Pasteur | Paris, |
| PCC 7507 | | | Culture | France |
| | | | Collection | |
| Nostoc sp. PCC | 95% | NC_019676.1 | Pasteur | Paris, |
| 7107 | | | Culture | France |
| | | | Collection | |
| Nostoc sp. PCC | 96% | NC_003272.1 | Pasteur | Paris, |
| 7120 | | | Culture | France |
| | | | Collection | |
| Anabaena | 96% | NC_007413.1 | American | Manassas, |
| variabilis ATCC | | | Type Culture | Virginia, |
| 29413 | | | Collection | USA |
| Anabaena | 97% | NC_019771.1 | Pasteur | Paris, |
| cylindrica PCC | | | Culture | France |
| 7122 | | | Collection | |
| Nostoc | 97% | NC_010628.1 | Pasteur | Paris, |
| punctiforme PCC | | | Culture | France |
| 73102 | | | Collection | |

Isolates having at least 95% sequence identity to
Tolypothrix distorta 16S SEQ ID NO: 4

| | | | | |
|---|---|---|---|---|
| Nodularia | 96% | NZ_CP007203.1 | Culture | Bratislava, |
| spumigena | | | collection of | Slovakia |
| CCY9414 | | | yeasts | |
| Calothrix sp. | 99% | NC_019682.1 | Pasteur | Paris, |
| PCC 7507 | | | Culture | France |
| | | | Collection | |
| Nodularia | 96% | CP007203.2 | Culture | Bratislava, |
| spumigena | | | collection of | Slovakia |
| CCY9414 | | | yeasts | |
| Cylindrospermum | 97% | NC_019757.1 | Pasteur | Paris, |
| stagnale PCC | | | Culture | France |
| 7417 | | | Collection | |
| Anabaena | 97% | NC_019771.1 | Pasteur | Paris, |
| cylindrica PCC | | | Culture | France |
| 7122 | | | Collection | |
| Nostoc | 97% | NC_010628.1 | Pasteur | Paris, |
| punctiforme PCC | | | Culture | France |
| 73102 | | | Collection | |
| Nostoc sp. PCC | 96% | NC_003272.1 | Pasteur | Paris, |
| 7120 | | | Culture | France |
| | | | Collection | |

¹Sequences of the whole genomes or a selected chromosome of the indicated strains that contain the 16S RNA encoding sequences can be obtained from the National Center Biotechnology Information National Center for Biotechnology Information (NCBI) with the indicated accession numbers via the internet at the world wide web site “ncbi.nlm.nih.gov/nuccore.” The NCBI is in the National Library of Medicine, Building 38A, Bethesda, MD 20894.
²American Type Culture Collection (ATCC) isolates can be accessed via the internet at the World Wide Web site “atcc.org.” The address for the ATCC is 10801 University Boulevard Manassas, VA 20110 USA.
Culture Collection of Yeasts (CCY) isolates can be accessed via the internet at the world wide web site “ccy.sk.” The address for the CCY is Institute of Chemistry, Slovak Academy of Sciences, Dúbravská cesta 9, 845 38 Bratislava, Slovakia.
Pasteur Culture collection of Cyanobacteria (PCC) isolates can be accessed via the internet at the http address “cyanobacteria.web.pasteur.fr/.” The address for the PCC is Collections des Cyanobactéries, Institut Pasteur, 28, rue du Docteur Roux, 75724 PARIS Cedex 15, FRANCE.

Ratios of the cyanobacteria within the composition or coating can be varied. For example, equal parts of each cyanobacterium species can be provided in the composition or coating. Alternatively, a 3:1:1:1 mixture by mass of Nostoc sp., Aulosira sp., Anabaena sp., and Tolypothrix sp., can be used.

Ratios with a range of about 5:1, 4.5:1, 4:1, 3.5:1, 3:1, 2.5:1, 2:1, 1.5:1, 1.1:1, or 1:1 to about 1:1.1, 1:1.5, 1:2, 1:2; 1:2.5; 1:3, 1:3.5, 1:4, 1:4.5, or 1:5 of (i) an Aulosira sp., to a Tolypothrix sp.; or (ii) an Aulosira sp. to an Anabaena sp. can be used.

Ratios with a range of about 5:1, 4.5:1, 4:1, 3.5:1, 3:1, 2.5:1, 2.4:1, 2.3:1, 2.2:1, 2.1:1, or 2:1 to about 1.9:1, 1.8:1, 1.7:1, 1.6:1, 1.5:1, 1.4:1, 1.3:1, 1.2:1 or 1.1:1 of (i) an Aulosira sp., to a Tolypothrix sp.; or (ii) an Aulosira sp. to a Anabaena sp. can be used. For example, the Aulosira species and the Tolypothrix species can be in a ratio of 2.2:1 to 1.8:1.

Ratios ranging from about 3:1, 2.9:1, 2.8:1, 2.7:1, 2.6:1, or 2.5:1 to about 1.9:1, 1.8:1, 1.7:1, 1.6:1, 1.5:1, 1.4:1, 1.3:1, 1.2:1 or 1.1:1 of an Aulosira sp., to a Tolypothrix sp. can be used.

Ratios of about 1:1:1 of an Aulosira sp., an Anabaena sp., and a Tolypothrix sp. can be used.

The cyanobacteria described herein can be used to provide a total of about 0.0001 milligram to 5 milligram (dry weight) per seed. For example, a total of about 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19 or 0.2 milligrams of the cyanobacterium in wet or dry weight per seed can be used. For example, a total of about 0.001 to about 0.05, 0.06, 0.07, 0.08, 0.09, or 0.1 milligram wet or dry weight of the cyanobacteria can be on the seed.

If a larger amount of coating per seed is desirable, from about 0.1, 0.2, or 0.3 to about 0.5, 0.6, 0.7, 0.8, 0.9, 1, 1.5, 2, 3, 4, or 5 milligrams per seed of the cyanobacterium in wet or dry weight can be used. For example, a total of 0.1 to about 0.9, 1, 1.5, 2, 3, 4, or 5 milligram wet or dry weight of the cyanobacteria can be on the seed.

The cyanobacterium can be provided at the equivalent of about 0.1, 0.2, 0.3, 0.4, or 0.5 kg to about 1, 1.1, 1.2, 1.3, 1.4, 1.5, 1.6, 1.7, 1.8, 1.9, 2, 2.5, 3, 3.5, 4, 4.5 or 5 kg wet weight per acre, or at the equivalent of about 12.5, 15, 20, 25, 30, 35, 40, 45, 50, 55, 60, 65, 70 or 75 grams to about 125, 150, 175, 200, 250, 300, 350, 400, 450 or 500 grams dry weight per acre of treated and sown seed.

Agriculturally acceptable adjuvants used in the compositions or coatings can include, but are not limited to, one or more insecticides, fungicides, safeners, biological fertilizers, water retention compounds, nutrient retention compounds, biochar or a combination thereof.

Such adjuvants can be selected for an absence of bacteriocidal, bacteriostatic, or bacterio-inhibitory activities that would reduce the effectiveness of the cyanobacteria provided in the composition or coating.

The insecticide can comprise a carbamate, an organophosphate, a neonicotinoid, a pyrethroid, or a combination thereof.

A neonicotinoid insecticide can comprise thiamethoxam, imidacloprid, clothianidin, nitenpyram, nithiazine, thiacloprid, or a combination thereof.

The fungicide can include one or more of an azole, strobilurin, or a metalaxyl compound, or a combination thereof. Useful azole fungicides include, but are not limited to, difenoconazole, prothioconazole, and tebuconazole. Useful strobilurin fungicides include, but are not limited to, kresoxim-methyl, azoxystrobin, trifloxystrobin, fluoxastrobin, picoxystrobin, pyraclostrobin, dimoxystrobin, pyribencarb, metominostrobin, orysastrobin, enestrobin, pyraoxystrobin and pyrametostrobin. Useful metalaxyl fungicides include, but are not limited to, metalaxyl and mefenoxam.

Agriculturally acceptable excipients used in the compositions or coatings can include, but are not limited to, one or more bulking agents, binding agents, colorants, emulsifiers, oils, tackifiers, trace elements, and/or extending agents.

Useful bulking agents include, but are not limited to, peat, wood flour, calcium carbonate, lime, diatomaceous earth, forms of clay such as bentonite and kaolin, and combinations thereof.

Useful binding agents can be water soluble polymers and/or waxes. Binding agents that are used can include, but are not limited to, polyvinyl acetate, polyvinyl alcohol, polyvinyl pyrrolidone, polyurethane, methyl cellulose, carboxymethyl cellulose, hydroxypropyl cellulose, sodium alginate, polyurethane, polyacrylate, casein, gelatin, pullulan, polyacrylamide, polyethylene oxide, polystyrene, styrene acrylic copolymers, styrene butadiene polymers, poly(N-vinylacetamide), and combinations thereof. Waxes used as binders can include, but are not limited to, carnauba wax, paraffin wax, polyethylene wax, bees wax, and polypropylene wax.

Emulsifiers can be either ionic or non-ionic agents. Emulsifiers that can be used include, but are not limited to, lecithin, polysorbates, polyethylene glycols, derivatives thereof, and the like.

Oils that can be used include, but are not limited to, silicon, animal, plant, or mineral oils and mixtures thereof.

An exogenous tackifier can be added to the composition or coating. However, content of an exogenous tackifier can be reduced in comparison to exogenous tackifier content of a standard microbial seed inoculant.

Extenders are materials that provide for improvements in the viability of cyanobacteria on the seed either pre or post planting. Useful extenders include, but are not limited to, trehalose, sucrose, glycerol, sorbitol, and combinations thereof. Liquid seed treatment inoculums containing various microorganisms and extenders comprising one or more of trehalose, sucrose, glycerol or sorbitol at about 5% to about 50% by weight/volume and related methods where a partially desiccated liquid inoculant product for application to seeds is prepared are described in U.S. Pat. No. 8,020,343 and can be adapted to use with the cyanobacteria compositions or coatings provided herein. U.S. Pat. No. 8,020,343 is incorporated herein by reference in its entirety. Other liquid seed treatment compositions that can be adapted for use with the cyanobacteria compositions or coatings provide herein can comprise sucrose, sorbitol, or a combination thereof at about 5% to 60% weight/volume, mineral oil or silicon oil at about 0.15% to about 3% weight, and an emulsifying agent selected from the group consisting of lecithin and polysorbate and are described in U.S. Pat. No. 8,551,913. U.S. Pat. No. 8,551,913 is incorporated herein by reference in its entirety.

Exopolysaccharides (EPS) can be produced by the cyanobacteria in the composition or coating (“endogenous” EPS), by cyanobacteria not in the composition or coating, or by other bacteria (“exogenous” EPS). Exogenous EPS can also be used as agriculturally acceptable excipients in the compositions and coatings provided herein.

The exogenous EPS can be provided in the composition or coating by simply adding fermentation broths, filtrates, supernatants, purified fractions, partially purified fractions, and the like that are obtained from cyanobacterial or other bacterial cultures. Without seeking to be limited by theory, it is believed that such EPS can improve water retention and/or desiccation tolerance of cyanobacteria in the compositions or coatings provided herein by slowing the desiccation process. It has been reported that bacterial EPS can help bacteria adapt to variable hydration conditions (Or et al. Advances in Water Resources, 30 (2007), pp. 1505-1527; Redmile-Gordon et al., Soil Biology & Biochemistry 72 (2014) 163e171). Exogenous exopolysaccharides obtained from sources other than cyanobacterial or other bacterial cultures that include, but are not limited to, fungal or yeast sources can be used either alone or in combination with the aforementioned cyanobacterial or bacterial cultures as agriculturally acceptable excipients.

Furthermore, a reduction in content of exogenous tackifier can be achieved from the endogenous EPS of the cyanobacteria of the composition or coating, or by using exogenous EPS as an agriculturally acceptable excipient in the composition or coating.

In the compositions and coatings described herein, water is not an excipient or adjuvant. To the extent that water content of a seed increases after it is coated and lyophilized or dried, the increase in water content is generally observed within the cyanobacteria itself.

The composition or coating can contain at least one element or a salt thereof comprising iron, boron, manganese, zinc, molybdenum, copper, cobalt, or a combination thereof.

The treated seeds can be used to improve: (i) biomass, yield, nutrient uptake and/or nitrogen uptake in a dicot plant including, but not limited to a soybean, a cotton, a Brassica sp., a tomato, a peanut, a biofuel crop, or a forage crop; (ii) plant growth in a dicot plant, including, but not limited to, improved growth in comparison to a dicot plant obtained from an untreated control seed where the plants are grown under conditions where nitrogen fertilizer is not used, a suboptimal amount of nitrogen fertilizer is used, or where less than 40, 50, 60, 70, 80, 90, 100, 150 or 200 lbs of nitrogen fertilizer per acre is used; or (iii) biomass, yield, nutrient uptake and/or nitrogen uptake in a dicot plant obtained from the dicot seed including, but not limited to, improvements in comparison to a dicot plant obtained from an untreated control dicot seed.

Yield resulting from the treated dicot plant seed can be at least 2.0% greater than yield resulting from the same dicot plant seed that is not treated with the coating when grown under greenhouse conditions. For example, yield resulting from the treated dicot plant seed can be at least 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, or 25.0% greater than yield resulting from the same dicot plant seed that is not treated with the coating when grown under greenhouse conditions as defined below. For example, yield can be increased by about 2-6% in soy and 3-15% in cotton.

Yields increases of as much as 35% can be obtained.

Nitrogen uptake resulting from the treated dicot plant seed can be at least 3% greater than nitrogen uptake resulting from the same dicot plant seed that is not treated with the coating when grown under the greenhouse conditions. For example, nitrogen uptake resulting from the treated dicot plant seed can be at least 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65 or 70% greater than nitrogen uptake resulting from the same dicot plant seed that is not treated with the coating when grown under the greenhouse conditions. For example, nitrogen uptake can be increased by about 3-20% in soy and about 5-40% in cotton.

Nitrogen uptake increases of as much as 80% can be obtained.

Nutrient uptake resulting from the treated dicot plant seed can be at least 2% greater than nutrient uptake resulting from the same dicot plant seed that is not treated with the coating when grown under the greenhouse conditions. For example, nutrient uptake resulting from the treated dicot plant seed can be at least 3, 4, 5, 6, 7, 8, 9, or 10% greater than nutrient uptake resulting from the same dicot plant seed that is not treated with the coating when grown under the greenhouse conditions.

Biomass resulting from the treated dicot plant seed can be at least 2% greater than biomass resulting from the same dicot plant seed that is not treated with the coating when grown under the greenhouse conditions. For example, biomass resulting from the treated dicot plant seed can be at least 3, 4, 5, 6, 7, 8, 9, 10, 11, 12, 13, 14, 15, 16, 17, 18, 19, 20, 21, 22, 23, 24, or 25% greater than biomass resulting from the same dicot plant seed that is not treated with the coating when grown under the greenhouse conditions. For example, biomass can be increased by about 2-10% in soy and about 5-20% in cotton.

Biomass increases of as much as 35% can be obtained.

Lateral root growth, lateral root hair growth, vertical root growth or root branching resulting from the treated dicot plant seed can exhibit increased branching as compared to lateral root growth, lateral root hair growth, vertical root growth or root branching resulting from the same dicot plant seed that is not treated with the coating when grown under the greenhouse conditions.

Also provided is a method of obtaining a dicot plant seed that provides for improved growth in a dicot plant obtained from the seed comprising applying a composition comprising the cyanobacteria to the at least a portion of an outer surface of the seed and lyophilizing or drying the composition to form the solid coating and obtain the treated dicot plant seed. The composition can be applied as a liquid or a slurry.

Also provided is a method of obtaining a plurality of dicot crop plants with improved yield, nitrogen uptake, nutrient uptake, biomass, lateral root growth, lateral root hair growth, vertical root growth or root branching, the method comprising: distributing the treated dicot plant seed on a plot of land and cultivating plants grown from the seed on the plot.

The methods can further comprise harvesting the cultivated plants or grain therefrom.

Also provided are methods of obtaining a dicot plant seed that provides for improved growth in a dicot plant obtained from the seed comprising applying a composition comprising: (i) at least one cyanobacterium species; and (ii) an agriculturally acceptable adjuvant, an agriculturally acceptable excipient, or combination thereof to at least one surface of the seed to obtain a seed that is at least partially coated with the composition, wherein growth of a dicot plant obtained from the treated dicot plant seed is increased in comparison to growth in a dicot plant obtained from an untreated control dicot plant seed.

In the methods described herein, the composition can be applied as a liquid or slurry.

The composition can be lyophilized or dried upon the surface of the seed.

Also provided is a method of improving yield, nitrogen uptake, nutrient uptake, biomass, lateral root growth, lateral root hair growth, vertical root growth or root branching in a dicot plant comprising:

exposing the dicot plant seed to an effective amount of a composition comprising cyanobacteria comprised of an Aulosira species and a Tolypothrix species, wherein either:

(1) the composition is free of an agriculturally acceptable adjuvant and/or an agriculturally acceptable excipient; or

(2) the composition further comprises the adjuvant and/or the excipient in an amount less than 0.4 kg/hectare; or

(3) the cyanobacteria consists essentially of the Aulosira species and the Tolypothrix species; or

(4) the composition consists essentially of the Aulosira species and the Tolypothrix species; or

(5) the composition comprises the Aulosira species and the Tolypothrix species in a weight ratio of about 5:1 to about 1:5; and allowing the dicot plant seed to germinate;

wherein either:

(A) yield resulting from the treated dicot plant seed is at least 2.0% greater than yield resulting from the same dicot plant seed that is not exposed to the composition when grown under greenhouse conditions; or

(B) nutrient uptake resulting from the treated dicot plant seed is at least 2% greater than nitrogen uptake resulting from the same dicot plant seed that is not exposed to the composition when grown under the greenhouse conditions;

(C) nitrogen uptake resulting from the treated dicot plant seed is at least 3% greater than nitrogen uptake resulting from the same dicot plant seed that is not exposed to the composition when grown under the greenhouse conditions; or

(D) biomass resulting from the treated dicot plant seed is at least 2% greater than biomass resulting from the same dicot plant seed that is not exposed to the composition when grown under the greenhouse conditions; or

(E) lateral root growth, lateral root hair growth, vertical root growth or root branching resulting from the treated dicot plant seed exhibits increased branching as compared to lateral root growth, lateral root hair growth, vertical root growth or root branching resulting from the same dicot plant seed that is not exposed to the composition when grown under the greenhouse conditions;

wherein the greenhouse conditions are as follows:

(a) seeds are planted in Captina silt loam soil with application of 40 lb/acre phosphate fertilizer and 20 lb/acre potassium fertilizer with 3 plants per pot for hybrid plant and 6 plants per pot for pureline plant;

(b) temperature maintained at 25-28° C., 50-70% relative humidity and natural light supplemented with fluorescent light at 35 μmol m⁻² s⁻² photosynthetically active radiation to provide a 14 h photoperiod; and

(c) plant grown to 50% heading then entire above-ground biomass is collected and dried, and total weight and nitrogen concentration can be measured.

Increases in yield, nitrogen uptake, nutrient uptake, biomass, lateral root growth, lateral root hair growth, vertical root growth and root branching can be as described above. For example, yield resulting from the treated dicot plant seed can be at least 2.0% greater (e.g., at least 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, or 25.0% greater) than yield resulting from the same dicot plant seed that is not exposed to the composition when grown under greenhouse conditions. Nitrogen uptake resulting from the treated dicot plant seed can be at least 3% greater (e.g., at least 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65 or 70% greater) than nitrogen uptake resulting from the same dicot plant seed that is not exposed to the composition when grown under the greenhouse conditions. Nutrient uptake resulting from the treated dicot plant seed can be at least 2% greater (e.g., at least 3, 4, 5, 6, 7, 8, 9, or 10% greater) than nutrient uptake resulting from the same dicot plant seed that is not treated with the coating when grown under the greenhouse conditions. Biomass resulting from the treated dicot plant seed can be at least 2% greater (e.g., at least 3, 4, 5, 6, 7, 8, 9, 10, 11, 12, 13, 14, 15, 16, 17, 18, 19, 20, 21, 22, 23, 24, or 25% greater) than biomass resulting from the same dicot plant seed that is not exposed to the composition when grown under the greenhouse conditions. Lateral root growth, lateral root hair growth, vertical root growth or root branching resulting from the treated dicot plant seed can exhibit increased branching as compared to lateral root growth, lateral root hair growth, vertical root growth or root branching resulting from the same dicot plant seed that is not exposed to the composition when grown under the greenhouse conditions.

In the methods described herein, the exposing step can comprise applying the composition to the seed by either in furrow application or by a soil drench application.

In the methods described herein, the exposing step can comprise applying the composition to a surface of the seed to obtain a seed that is at least partially coated with the composition.

Also provided are methods of improving plant growth in a dicot plant comprising exposing a seed to an effective amount of a composition comprising: (i) at least one cyanobacterium species; and (ii) an agriculturally acceptable adjuvant, an agriculturally acceptable excipient, or combination thereof and allowing the seed to germinate, wherein growth of a dicot plant obtained from the seed exposed to the composition is increased in comparison to growth of a dicot plant obtained from a control dicot plant seed that was not exposed to the composition.

In the methods described herein, the seed can be exposed to a composition or the composition can be applied to the seed, the composition comprising: (i) an Aulosira species and a Tolypothrix species at a ratio of 3:1 to 1.5:1, respectively; and (ii) an agriculturally acceptable adjuvant, an agriculturally acceptable excipient, or a combination thereof.

Also provided are methods of improving biomass, yield, nutrient uptake and/or nitrogen uptake in a dicot plant, the method comprising exposing a dicot seed to an effective amount of a composition comprising: (i) at least one cyanobacterium species; and (ii) an agriculturally acceptable adjuvant, an agriculturally acceptable excipient, or combination thereof and allowing the seed to germinate, wherein biomass, yield, nutrient uptake and/or nitrogen uptake in a dicot plant obtained from the dicot seed exposed to the composition is increased in comparison to biomass, yield, nutrient uptake and/or nitrogen uptake in a dicot plant obtained from a control dicot seed that was not exposed to the composition.

Any method of measuring the growth characteristic can be employed. Typically, the biomass, yield, and the like can be assessed by determining the mass of plant material obtained, the mass of seeds obtained, or the number of seeds and a per plant or per unit area (e.g., per acre or hectare) basis. Lateral root growth, lateral root hair growth, vertical root growth, or root branching can be measured by visual inspection to determine length and/or width or by imaging to quantify the length, width, density, volume or branching of roots and root systems.

Seeds treated with compositions or coatings provided herein can be used to improve dicot plant growth under nitrogen limiting conditions and/or to improve nitrogen uptake by dicot plants. Plant growth can improve under conditions where nitrogen is limiting. When a suboptimal amount of nitrogen fertilizer is used, a minimum of 0.1 lb/acre can be used.

The plant growth medium in which the plant seed is grown need not be supplemented with nitrogen if desired.

In the methods described herein, it may be desirable not to use nitrogen fertilizer, or to use a suboptimal amount of nitrogen fertilizer. For example, less than 5, 10, 15, 20, 25, 30, 35, 40, 45, 50, 60, 70, 80, 90, 100, 110, 120, 130, 140, 150, 160, 170, 180, 190 or 200 lbs/acre of nitrogen fertilizer can be used in the methods.

Where nitrogen fertilizer is not used or where a suboptimal amount of nitrogen fertilizer is used, compositions or coatings comprising: (i) an Aulosira sp., and a Tolypothrix sp.; (ii) an Aulosira sp.; (iii) an Aulosira sp. and an Anabaena sp.; (iv) an Aulosira sp., an Anabaena sp., and a Tolypothrix sp.; or (v) a Nostoc sp. can be used.

Where nitrogen fertilizer is not used or where a suboptimal amount of nitrogen fertilizer is used, compositions or coatings comprising (i) Aulosira bohemensis and Tolypothrix distorta; (ii) Aulosira bohemensis; (iii) Aulosira bohemensis and Anabaena cylindrica; (iv) an Aulosira bohemensis, an Anabaena cylindrica sp., and a Tolypothrix distorta sp.; or (v) a Nostoc commune sp. can be used.

Where nitrogen fertilizer is not used or where a suboptimal amount of nitrogen fertilizer is used, compositions or coatings comprising (i) Aulosira bohemensis and Tolypothrix distorta; (ii) Aulosira bohemensis; (iii) Aulosira bohemensis and Anabaena cylindrica; (iv) an Aulosira bohemensis, an Anabaena cylindrica sp., and a Tolypothrix distorta sp.; or (v) a Nostoc commune sp. can be used. The amount of nitrogen fertilizer used is usually less than an equivalent of 110 lbs/acre of nitrogen.

Improvements in dicot plant growth or dicot plant yield obtained by seed treatments with cyanobacterial compositions or coatings provided herein under any of the aforementioned nitrogen limiting conditions can be at least about 3, 4, 9, 10, 15, or 20% in comparison to an untreated control dicot plant or can be about 3, 4, 5, or 10% to about 15, 18, 20, 30, or 40% in comparison to an untreated control dicot plant.

Improvements in dicot plant growth or dicot plant yield under any of the aforementioned nitrogen limiting conditions with a cyanobacterial seed treatment provided herein can be at least about 10% in comparison to an untreated control dicot plant or can be about 10, 15, 18, or 25% in comparison to a dicot plant subjected to a topical treatment of the cyanobacteria.

Nitrogen uptake by plants obtained from seeds treated with the cyanobacterial compositions or coatings provided herein can be improved under nitrogen limiting conditions and under conditions where nitrogen is not limiting. Nitrogen uptake can be determined by a variety of methods that include, but are not limited to, isotopic and non-isotopic methods that have been described (Sandrock et a. Hort. Sci. 40(3):665, 2005; Norman et al. Soil Sci. Soc. Am. J. 56:1521-1527. 1992).

Improvements in nitrogen uptake by dicot plants obtained by seed treatments with cyanobacterial compositions or coatings provided herein under any of the aforementioned nitrogen limiting conditions can be at least about 3, 4, 5, 10, 20, 40, 50, or 60% in comparison to an untreated control dicot plant or can be about 3, 4, 5, or 10%, to about 15, 18, 20, 40, 60, or 70% in comparison to an untreated control dicot plant.

Improvements in nitrogen uptake under any of the aforementioned nitrogen limiting conditions with a cyanobacterial seed treatment provided herein can be at least about 3, 4, 9, or 10% in comparison to an untreated control dicot plant or can be about 3, 4, or 5% to about 10, 15, 20, 30, or 40% in comparison to a dicot plant subjected to a topical treatment of the cyanobacteria.

In the methods and seed treatments described herein, the seed can be dormant before, during, and/or after treatment.

The dicot plant or dicot plant seed can comprise soybean, cotton, Brassica sp., tomato, peanut, a biofuel crop, or a forage crop. The Brassica sp. plant or seed can be a Brassica napus (e.g., a canola seed), or Brassica oleracea. The forage crop plant or seed can be an alfalfa, clover, or vetch. The biofuel crop can be Camelina, Jatropha, sunflower, or flax.

Seed coating equipment and associated techniques used to coat the seeds include, but are not limited to, drum coaters, fluidized beds, rotary coaters, side vended pan, tumble mixers, and spouted beds. Conventional seed coating methods for use herein are well known in the art.

### Definitions

Any ranges described herein in the form of “A, B, or C to X, Y, or Z” means that every possible combination of such ranges are disclosed herein (i.e., A to X, A to Y, A to Z, B to X, B to Y, B to Z, C to X, C to Y, and C to Z). For example, a range of “about 3%, 4%, or 5% to about 10%, 15%, 20%, 30%, or 40%” means that the ranges “about 3% to about 10%, about 3% to about 15%, about 3% to about 20%, about 3% to about 30%, about 3% to about 40%, about 4% to about 10%, about 4% to about 15%, about 4% to about 20%, about 4% to about 30%, about 4% to about 40%, about 5% to about 10%, about 5% to about 15%, about 5% to about 20%, about 5% to about 30%, and about 5% to about 40%” are described. As another example, a range of ratios of “about 3:1, 2.5:1, or 2.2:1 to about 1.8:1, 1.7:1, or 1.5:1” means that the ranges “about 3:1 to about 1.8:1, about 3:1 to about 1.7:1, about 3:1 to about 1.5:1, about 2.5:1 to about 1.8:1, about 2.5:1 to about 1.7:1, about 2.5:1 to about 1.5:1, about 2.2:1 to about 1.8:1, about 2.2:1 to about 1.7:1, about 2.2:1 to about 1.5:1” are described.

The term “consisting essentially of” as used herein means that the composition, coating or cyanobacteria contains the specified ingredients, as well as additional unspecified ingredients, provided that the unspecified ingredients do not materially affect the basic and novel characteristics of the composition, coating, or cyanobacteria. These characteristics include increased yield, nitrogen uptake, nutrient uptake, biomass, lateral root growth, lateral root hair growth, vertical root growth and/or root branching as compared to seeds untreated with the composition, coating or cyanobacterium under greenhouse conditions as specified herein. The composition, coating or cyanobacterium excludes, for example, an amount of any cyanobacterium species (such as Nostoc species) that would decrease yield, nitrogen uptake, nutrient uptake, biomass, lateral root growth, lateral root hair growth, vertical root growth and/or root branching as compared to the same composition, coating or cyanobacteria that does not include the cyanobacterium species when grown under the specified greenhouse conditions.

As used herein in reference to a cyanobacterium preparation, “dry weight” refers to the weight of cyanobacterium that have been both separated from liquid and dried. Such drying can be effected by methods including, but not limited to, evaporation, lyophilization, or combinations thereof.

“Endogenous EPS” is EPS produced by any cyanobacterium within the composition or coating.

“Exogenous EPS” is EPS produced by any cyanobacterium not in the composition or coating, or produced by bacteria other than a cyanobacterium.

When “greenhouse conditions” are specified for comparative results herein, the conditions are as follows when using treated seeds:

(a) seeds are planted in Captina silt loam soil with application of 40 lb/acre phosphate fertilizer and 20 lb/acre potassium fertilizer with 3 plants per pot for hybrid plant and 6 plants per pot for pureline plant;
(b) temperature maintained at 25-28° C., 50-70% relative humidity and natural light supplemented with fluorescent light at 35 μmol m⁻² s⁻² photosynthetically active radiation to provide a 14 h photoperiod; and
(c) plant grown to 50% heading then entire above-ground biomass is collected and dried, and total weight and nitrogen concentration can be measured.

As used herein, the term “lateral root” refers to a root that originates from a primary or nodal root.

As used herein, the term “lateral root hair” refers to a root hair that originates from a lateral root.

As used herein, the terms “nitrogen limiting conditions” or “a suboptimal amount of nitrogen fertilizer is used” refer to conditions where plant growth or yield can be increased upon supplementing the plant growth medium (e.g., soil, synthetic rooting mixes, hydroponic liquids, flooded fields, and the like) with exogenous nitrogen or by improving the availability of nitrogen already present in the plant growth medium.

A “nutrient” is defined as a mineral or inorganic compound that is absorbed from soil or other growth medium that is essential for plant growth or metabolism. Nutrients include, but are not limited to, nitrogen, phosphorus, potassium, calcium, sulfur, magnesium, boron, chlorine, manganese, iron, zinc, copper, molybdenum, and nickel.

“Root branching” is defined herein as the process in which lateral roots grow out from the taproot and other lateral roots for the purpose of providing plant stability and nutrient uptake.

“Vertical root growth” is defined herein as the elongation of the root, taproot, and seminal roots in a direction perpendicular to the soil surface.

As used herein in reference to a cyanobacterium preparation, “wet weight” refers to the weight of cyanobacterium that have been separated from liquid but not dried. Such separation can be effected by methods including, but not limited to, settling/decanting, filtration, centrifugation, or combinations thereof.

Unless otherwise defined, all technical and scientific terms used herein have the same meaning as commonly understood by one of ordinary skill in the art to which this disclosure pertains. In the case of inconsistencies between the present disclosure and the issued patents, applications, and references that are cited herein, the present disclosure will prevail.

Having described the invention in detail, it will be apparent that modifications and variations are possible without departing from the scope of the invention defined in the appended claims.

### EXAMPLES

The following non-limiting examples are provided to further illustrate the present invention.

### Example 1. Microbial Biomass Production

Nostoc commune UTEX B 1621, Anabaena cylindrica UTEX B 1611, Aulosira bohemensis UTEX B 2947, and Tolypothrix distorta UTEX 424 will be maintained under constant shaded cool white fluorescent light on sterile BG11 plates. BG11 media has been described (Rippka Archiv für Mikrobiologie 87, Issue 1, pp 93-98, 1972) and is commercially available (Sigma-Aldrich, St. Louis, Mo.). Plates will be re-streaked onto fresh plates as colonies appear using a microbiological loop under sterile conditions. For amplification, biomass from plates will be transferred into several autoclaved 250 ml Erlenmeyer flasks containing 100 ml sterile BG11 by swishing a sterile microbiological loop containing microbial biomass (obtained from the plates) in the liquid media. The Erlenmeyer flasks will be stoppered with autoclaved foam stoppers.

The 250 ml Erlenmeyer flasks containing monocultures will be shaken at 120 rpm on an orbital shaker in constant cool white fluorescent light at 22° C. until cultures reach translucent green cell density. The entirety of four 250 ml Erlenmeyer flasks with the same species of cyanobacteria, containing 100 ml of BG11 each, will then be transferred to autoclaved tubular 4 L flasks containing 3.5 L sterile BG11. The 4 L bubble flasks will be mixed by aeration through an autoclaved bubble tube connected to an aquarium pump. A filter made of autoclaved cotton fibers will be placed in the tubing line to maintain sterility. An autoclaved foam stopper with a hole for the air-in port will be used to seal the 4 L flasks. The culture will be supplied with constant cool white fluorescent light at 22° C.

Upon reaching translucent green cell density, 2 L culture from the 4 L bubble flasks will be transferred to a 20 L carboys containing 14 L sterile BG11 (for a total culture volume of 16 L). The 4 L bubble flask will be replenished with 2 L sterile BG11 to allow for additional 20 L inoculations as necessary. Monocultures growing in 20 L carboys will be diluted by half with fresh sterile BG11 as necessary to maintain dense, translucent green cultures. As necessary, upon dilution with fresh BG11, excess culture will be transferred to plastic 20 L carboys that are washed with antibacterial soap and sterilized with ethanol. These cultures will also be supplied with constant light at 22° C. If necessary to generate appropriate biomass, 20 L carboys will be used to inoculate 100 L flat panel photobioreactors containing 80 L sterile BG11.

Harvesting of cyanobacterial monocultures will begin by settling monocultures growing in 20 L carboys or 100 L photobioreactors for 2-4 hours (total settling time depended on time it takes individual containers to settle). After settling, clear supernatant will be siphoned off using autoclaved plastic tubing. A total of 1-6 L of each monoculture per 20 L carboy will remain after settling. Settled cultures will then be pulse blended in an Oster blender for less ˜5 seconds. Cultures will then be centrifuged at 6,000×g for 10 minutes in either a 6×50 ml benchtop centrifuge or a 6×1 L floor centrifuge. Centrifuge size will be chosen based upon total biomass required for subsequent plant treatments. Each vessel will be decanted after centrifugation, and biomass pellets will be combined into a sterilized beaker. Total wet weight biomass will be determined by measuring the weight of the beaker before and after the addition of biomass. Biomass cyanobacterial mixtures will be prepared by mixing biomass paste of each monoculture by weight with the other members of the cyanobacterial mixture to give the prescribed ratio. Biomass paste will then be diluted to a working volume with sterile Millipore water, giving a known working biomass concentration.

### Example 2. Dicot Seed Coating

A total of 4 mg cyanobacteria will be applied per seed to dicot seeds (e.g. soybean, cotton, tomato, Brassica sp., a peanut, biofuel crop, or a forage crop seeds) in batch applications. A control will be also created with no cyanobacteria applied. Cyanobacterial biomass will be prepared by harvesting excess liquid cyanobacterial monoculture, centrifuging, and removing the supernatant. The culture will then be centrifuged in tubes for 10 min at 6000×g. The biomass pellets will then be diluted with a small amount of Nanopure™ water (Thermo Fischer Scientific, Inc. USA), harvested and combined into one tube. The cyanobacterial culture will then be homogenized by blending on a pulse setting in a kitchen blender for less than five seconds. As little blending as possible will be used to prevent unnecessary cell damage. After homogenization, the biomass will be spun for 10 min at 6000×g to remove any remaining liquid. Cyanobacterial biomass will be added to individual tubes as a paste. Biomass will be calculated by subtracting the weight of the tube and paste from the weight of the tube only (determined at beginning of experiment). The 0 and 4 mg per seed application rates will then be diluted with Nanopure™ water to a known total volume before coating. Six replicates of each application rate will be prepared. Four will be used in seed coating and two will serve as backups in case there are unforeseen issues with coating.

Dicot seeds will be coated with 4 mg per seed of cyanobacteria (prepared as above) using Hege™ 11 liquid seed treater. Coating will be performed in 3 applications. Between coatings, seeds will be allowed to dry on a cookie sheet at room temperature for four hours. Drying between applications will be performed to prevent early germination.

### Example 3. Germination and Root Hair Formation

Dicot seeds (e.g. soybean, cotton, tomato, Brassica sp., a tomato, a peanut, a biofuel crop, or a forage crop seeds) will be germinated and images of the roots will be captured daily for one week. Changes in the formation of lateral root hairs will be measured.

### Example 4. Greenhouse Testing of Cyanobacterium Treated Seed

A greenhouse trial will be performed with plants grown from treated and untreated control dicot seeds (e.g. peanut, soybean, cotton, tomato, Brassica sp., a tomato, a biofuel crop, or a forage crop seeds). Mixture A will be Nostoc commune UTEX B1621 alone. Mixture B will be a 3:1:1:1 mixture of Nostoc commune UTEX B 1621, Anabaena cylindrica UTEX B 1611, Aulosira bohemensis UTEX B 2947, and Tolypothrix distorta UTEX 424. Mixture C will be a 1:1:1 mixture of Aulosira bohemensis UTEX B 2947, Anabaena cylindrica UTEX B 1611, and Tolypothrix distorta UTEX 424. All cyanobacterial mixtures will be applied at a rate of 4 mg cyanobacterial mixture per seed. During the trial, seeds coated with Mixture A, Mixture B, Mixture C, and a control with no seed coating (Table 2) will be grown for 8-16 weeks in a greenhouse. Plants will be grown in plastic pots in potting soil. They will be watered in four stepwise chemical nitrogen regimes which represent the typical amounts applied in commercial agricultural operations. Physiological parameters that include, but are not limited to: chlorophyll content, biomass, stem diameter and leaf formation will be measured. It is anticipated that seeds coated with Mixture A, Mixture B, or Mixture C will provide dicot plants with improved growth characteristics when compared to dicot plants obtained from control seeds with no seed coating.

### Example 5. Cyanobacterial Treatment of Dicot Plants by Treatment of Fields or by Seed Treatment in Field Tests

A mixture of Nostoc commune UTEX B 1621, Aulosira bohemensis UTEX B 2947, Anabaena cylindrica UTEX B 1611, and Tolypothrix distorta UTEX 424 respectively will be maintained and harvested essentially as described in Example 1. The cyanobacterial mixture will be diluted to a working concentration that allows the biomass to be treated as a liquid. Seed coating will be carried out in a Hege™ 11 Seed Coater. A desired coating rate of 500 g cyanobacterial mixture per acre will be achieved. This cyanobacterial mixture will be applied in three separate aliquots, and seeds will be allowed to dry before applying additional cyanobacterial mixture. After all coating runs, seeds will be spread out into a single layer and allowed to dry overnight at room temperature in a well-ventilated area.

Dicot seeds (e.g. soybean, cotton, tomato, Brassica sp., a tomato, peanut, a biofuel crop, or a forage crop seeds) treated as described above will then be planted in fields and grown in 0, low (˜75), and high (˜150) lbs/acre nitrogen. Upon reaching full maturity, dicot plants will be harvested and yield and total nitrogen uptake will be measured. Total above ground biomass will be collected from a 1 m section of the first bordered row. Following maturity, plots will be harvested. Statistical analysis will be conducted using a standard ANOVA and means were separated using Fisher's protected LSD at the α=0.05 level where appropriate.

### Example 7. Greenhouse Testing of Cyanobacterium Treated Seed

A greenhouse trial was performed with plants grown from treated and untreated control cotton seeds. The Mixture was a 2:1 ratio of Aulosira bohemensis UTEX B 2947 and Tolypothrix distorta UTEX 424. It was applied at a rate of 4 mg cyanobacterial mixture per seed. The cotton cultivar Stoneville 4946 GLB2 was used, as it is currently the most commonly used cultivar in the United States.

The study was implemented as a randomized complete block design with a factorial arrangement of two soils (Crevasses and Loring) and four N treatments (Nitrogen-Treatment). The four N-Treatments were: 1) 0 Cyanobacteria, 0 N fertilizer (control); 2) 500 g/acre dosage of cyanobacteria, 0 synthetic N fertilizer; 3) 0 Cyanobacteria, urea to meet half of crop's N requirement to simulate the pre plant N application rate; and 4) no Cyanobacteria, urea to meet all of the crop's N requirement.

Four different fertilization levels were tested in the trial. Control seeds, with no coating and no additional fertilizer; seeds coated with Mixture; uncoated seeds with nitrogen fertilizer (46-0-0 NPK) equal to a 90 lbs per acre; and uncoated seeds with nitrogen fertilizer (46-0-0 NPK) equal to 45 lbs per acre. These were grown for 98 days in a greenhouse. Two types of soil were tested, Crevasses and Loring. Soil samples were dug up from research plots at the University of Arkansas, sieved and added to plastic pots. Soil pH, organic matter, NO₃—N, and Mehlich-3 extractable nutrients were measured by standard methods. Soil texture was measured by the hydrometer method.

Three cotton seeds were planted in each pot and thinned to one seed per pot five days after emergence. Supplemental lights were provided for 12 hours from 7:00 AM to 7:00 pm. The greenhouse temperature was maintained at no lower than 68° F.

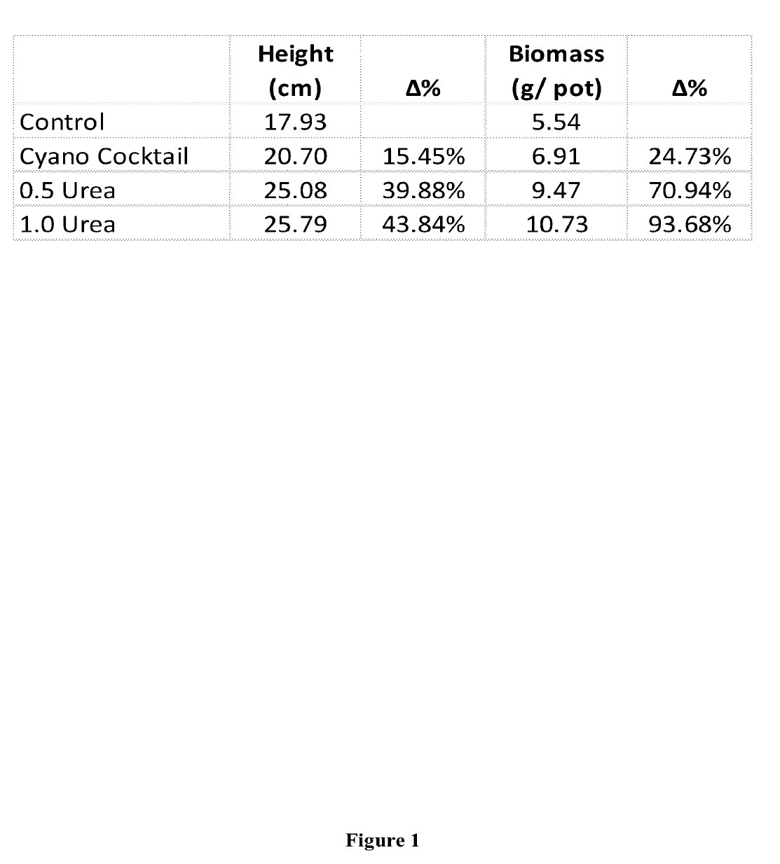

Analysis of variance (ANOVA) was performed to evaluate the effect of soil type, N-Treatment and their interaction on early cotton growth and development parameters by using the SAS Package. When appropriate, means were separated by the least significant difference (LSD) method and interpreted as significant when P≤0.10. Results are found in FIG. 1.

A noticeable difference in cotton growth and development between the two soils where the plants grew faster in Loring soil as compared to the Crevasses was observed, which was confirmed by statistical analysis of the data. Soil type and N-Treatment both significantly (P 0.1) influenced the total number of nodes, monopodial (vegetative) nodes, sympodial (fruiting nodes), average internode length (the distance between the two successive nodes), final plant height, and final dry biomass. However, the interaction of soil type by N-Treatment significantly influenced only dry biomass per plant. Plant height and dry weight per pot of the cotton grown in Loring soil were 70% and 140% more than the plants in the Crevasses soil.

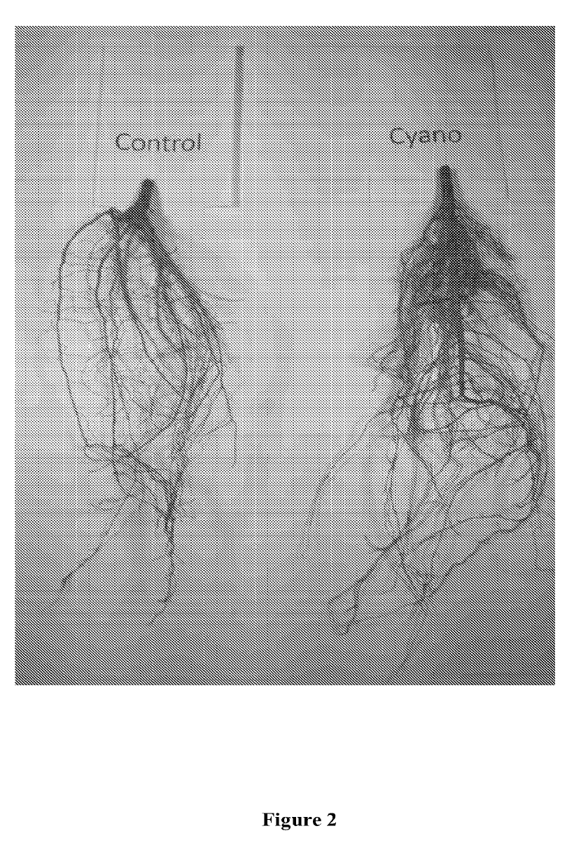

Visual evidence of root stimulation was observed. Seeds which had been treated with Mixture showed significantly more branching and deeper, thicker roots. Results are shown in in FIG. 2.

In Crevasses soil, cotton fertilized with the full N-rate produced significantly higher biomass than cotton treated with cyano bacteria or cotton that did not receive any N. However, there was no significant difference in biomass production among the all treatments that received less than the full N rate. Biomass production rate in Crevasses may have been limited by the low soil pH. Cotton fertilized with 0.5×N-rate produced numerically higher biomass than the cyano treated cotton which produced numerically higher biomass than the cotton that did not receive any cyano bacteria. In Loring soil cotton fertilized with the full N rate produced the highest dry biomass and cotton that did not receive any N or cyano bacteria produced the lowest biomass dry weight. The amount of biomass per single plant produced by the cyano treated bacteria was 25% more and significantly higher than the cotton that did not receive any N fertilizer or cyanobacteria.

The breadth and scope of the present disclosure should not be limited by any of the above-described exemplary embodiments, but should be defined only in accordance with the following claims and their equivalents. As various changes could be made in the above compositions and methods without departing from the scope of the invention, it is intended that all matter contained in the above description and shown in the accompanying drawings shall be interpreted as illustrative and not in a limiting sense.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

1. A treated dicot plant seed comprising a solid coating on at least a portion of an outer surface of the seed, the coating comprising cyanobacteria comprised of an Aulosira species and a Tolypothrix species, wherein either:
(1) the coating is free of an agriculturally acceptable adjuvant and/or an agriculturally acceptable excipient; or
(2) the coating further comprises the adjuvant and/or the excipient in an amount less than 0.09 gram per gram of seed; or
(3) yield resulting from the treated dicot plant seed is at least 2.0% greater than yield resulting from the same dicot plant seed that is not treated with the coating when grown under the following greenhouse conditions:
(a) seeds are planted in Captina silt loam soil with application of 40 lb/acre phosphate fertilizer and 20 lb/acre potassium fertilizer with 3 plants per pot for hybrid plant and 6 plants per pot for pureline plant;
(b) temperature maintained at 25-28° C., 50-70% relative humidity and natural light supplemented with fluorescent light at 35 μmol m⁻² s⁻² photosynthetically active radiation to provide a 14 h photoperiod; and
(d) plant grown to 50% heading then entire above-ground biomass is collected and dried, and total weight and nitrogen concentration can be measured; or
(4) the cyanobacteria consists essentially of the Aulosira species and the Tolypothrix species; or
(5) the coating consists essentially of the Aulosira species and the Tolypothrix species; or
(6) the coating comprises the Aulosira species and the Tolypothrix species in a weight ratio of about 5:1 to about 1:5.

2. The treated dicot plant seed of claim 1, wherein the coating is free of an agriculturally acceptable adjuvant or an agriculturally acceptable excipient.

3. The treated dicot plant seed of claim 1, wherein the coating is free of an agriculturally acceptable adjuvant and an agriculturally acceptable excipient.

4. The treated dicot plant seed of claim 1, wherein the coating further comprises the adjuvant and/or the excipient in an amount less than 0.08, 0.07, 0.06, 0.05, 0.04, 0.03, 0.02, or 0.01 gram per gram of seed.

5-11. (canceled)

12. The treated dicot plant seed of claim 1, wherein the coating comprises the adjuvant and/or the excipient in an amount of at least 0.001 gram per gram of seed.

13. The treated dicot plant seed of claim 1, wherein the coating further comprises the cyanobacterium in an amount from about 0.001 g to about 0.15 gram per gram of seed.

14. The treated dicot plant seed of claim 13, wherein the coating further comprises the cyanobacterium in an amount less than 0.08, 0.09, 0.10, 0.11, 0.12, 0.13, or 0.14 gram per gram of seed.

15. (canceled)

16. The treated dicot plant seed of claim 1, wherein at least one of the following:
yield resulting from the treated dicot plant seed is at least 2.0, 3.0, 4.0, 5.0, 6.0, 7.0, 8.0, 9.0, 10.0% greater than yield resulting from the same dicot plant seed that is not treated with the coating when grown under the greenhouse conditions; and/or
nitrogen uptake resulting from the treated dicot plant seed is at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65 or 70% greater than nitrogen uptake resulting from the same dicot plant seed that is not treated with the coating when grown under the greenhouse conditions; and/or
biomass resulting from the treated dicot plant seed is at least 2, 3, 4, 5, 6, 7, 8, 9, 10, 11, 12, 13, 14, 15, 16, 17, 18, 19, 20, 21, 22, 23, 24, or 25% greater than biomass resulting from the same dicot plant seed that is not treated with the coating when grown under the greenhouse conditions; and/or
lateral root growth, lateral root hair growth, vertical root growth or root branching resulting from the treated dicot plant seed exhibits increased branching as compared to lateral root growth, lateral root hair growth, vertical root growth or root branching resulting from the same dicot plant seed that is not treated with the coating when grown under the greenhouse conditions; and/or
nutrient uptake resulting from the treated dicot plant seed is at least 2, 3, 4, 5, 6, 7, 8, 9, or 10% greater than nutrient uptake resulting from the same dicot plant seed that is not treated with the coating when grown under the greenhouse conditions.

17-23. (canceled)

24. The treated dicot plant seed of claim 16, wherein at least one of the following:
the treated dicot plant seed is soy, and either: yield is increased by 2-6%; nitrogen uptake is increased by about 3-20%; or nutrient uptake is increased by about 2-10%; and/or
the treated dicot plant seed is cotton, and either: yield is increased by about 32-15%; nitrogen uptake is increased by about 5-40%; biomass is increased by about 5-20%; or nutrient uptake is increased by about 2-10%; and/or
plant growth medium in which the plant seed is grown is supplemented with less than 10, 15, 20, 25, 30, 35, 40, 45, 50, 100 or 200 lbs nitrogen fertilizer/acre.

25-29. (canceled)

30. The treated dicot plant seed of claim 24, wherein either: the plant growth medium in which the plant seed is grown is supplemented with at least 0.1 lb nitrogen fertilizer/acre; or the plant growth medium in which the plant seed is grown is not supplemented with nitrogen.

31. (canceled)

32. The treated dicot plant seed of claim 1, wherein the cyanobacteria consists essentially of the Aulosira species and the Tolypothrix species; or
the coating consists essentially of the Aulosira species and the Tolypothrix species; or
the coating comprises the Aulosira species and the Tolypothrix species in a weight ratio of about 5:1 to about 1:5, about 3:1 to about 1:3, about 3:1 to 1.1:1, about 3:1 to about 1.5:1, about 2.5:1 to about 1.5:1, or about 2.2:1 to about 1.8:1.

33-39. (canceled)

40. The treated dicot plant seed of claim 1, wherein:
the dicot plant seed comprises soybean, cotton, Brassica sp., tomato, peanut, a biofuel crop, or a forage crop; or
the dicot plant seed comprises the Brassica sp. seed, and the Brassica sp. seed comprises a Brassica napus, or Brassica oleracea; or
the dicot plant seed comprises the forage crop seed, and the forage crop seed comprises an alfalfa, clover, or vetch; or
the dicot plant seed comprises the biofuel crop seed, and the biofuel crop seed comprises Camelina, Jatropha, sunflower, or flax.

41-43. (canceled)

44. The treated dicot plant seed of claim 1, wherein at least one of the following:
the Aulosira species is characterized by having a gene encoding a 16S RNA that has at least 95%, 97%, 98%, 99%, 99.5%, 99.7%, 99.9%, or 100% sequence identity across the entire length of SEQ ID NO:1; and/or
the Tolypothrix species is characterized by having a gene encoding a 16S RNA that has at least 95%, 97%, 98%, 99%, 99.5%, 99.7%, 99.9%, or 100% sequence identity across the entire length of SEQ ID NO:4; and/or
the Aulosira species comprises an Aulosira bohemensis UTEX B 2947 culture or variant thereof or wherein the Tolypothrix species comprises a Tolypothrix distorta UTEX 424 culture or a variant thereof; and/or
the Aulosira species comprises an Aulosira bohemensis UTEX B 2947 culture or variant thereof and wherein the Tolypothrix species comprises a Tolypothrix distorta UTEX 424 culture or a variant thereof; and/or
0.0001 milligram to 5 milligram (dry weight) of the Aulosira species and a Tolypothrix species is provided on the seed; and/or
less than 0.0005, 0.001, 0.005, 0.01, 0.02, 0.03, 0.04, 0.05, 0.06, 0.07, 0.08, 0.09, 0.10, 0.11, 0.12, 0.13, 0.14, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, or 4.5 milligram wet or dry weight of the Aulosira species and a Tolypothrix species is provided on the seed; and/or
wherein the agriculturally acceptable adjuvant comprises an insecticide, a fungicide, a safener, a biological fertilizer, a water retention compound, a nutrient retention compound, biochar or a combination thereof; and/or
wherein the agriculturally acceptable adjuvant comprises an insecticide, and the insecticide comprises a carbamate, an organophosphate, a neonicotinoid, a pyrethroid, or a combination thereof; and/or
wherein the agriculturally acceptable adjuvant comprises a neonicotinoid insecticide, and the neonicotinoid insecticide comprises thiamethoxam, imidacloprid, clothianidin, nitenpyram, nithiazine, thiacloprid, or a combination thereof; and/or
the coating contains at least one element or a salt thereof comprising iron, boron, manganese, zinc, molybdenum, copper, cobalt, or a combination thereof; and/or
the Aulosira species and the Tolypothrix species are not associated with the dicot plant seed in nature; and/or
the coating comprises an exogenous exopolysaccharide; and/or
the coating is lyophilized; and/or
the cyanobacteria does not further comprise an Anabaena species, a Nostoc species, or both an Anabaena species and a Nostoc species; and/or
the cyanobacteria does not further comprise Tolypothrix tenuis, Aulosira fertilissima, and a Nostoc species; and/or
the cyanobacteria does not further comprise Tolypothrix tenuis, Aulosira fertilissima, a Nostoc species, and a Cylindrospermum species.

45-58. (canceled)

59. A method of obtaining a plurality of dicot crop plants with improved grain yield, nitrogen uptake, nutrient uptake, biomass, lateral root growth, lateral root hair growth, vertical root growth or root branching, the method comprising: distributing the treated dicot plant seed of claim 1 on a plot of land and cultivating plants grown from the seed on the plot.

60. (canceled)

61. A method of obtaining a dicot plant seed that provides for improved growth in a dicot plant obtained from the seed comprising applying a composition comprising the cyanobacteria to the at least a portion of an outer surface of the seed and lyophilizing or drying the composition to form the solid coating and obtain the treated dicot plant seed of claim 1.

62. (canceled)

63. A method of improving biomass, yield, nutrient uptake, nitrogen uptake, lateral root growth, lateral root hair growth, vertical root growth and/or root branching in a dicot plant comprising:
exposing the dicot plant seed to an effective amount of a composition comprising cyanobacteria comprised of an Aulosira species and a Tolypothrix species, wherein either;
(1) the composition is free of an agriculturally acceptable adjuvant and/or an agriculturally acceptable excipient; or
(2) the composition further comprises the adjuvant and/or the excipient in an amount less than 0.4 kg/hectare; or
(3) the cyanobacteria consists essentially of the Aulosira species and the Tolypothrix species; or
(4) the composition consists essentially of the Aulosira species and the Tolypothrix species; or
(5) the composition comprises the Aulosira species and the Tolypothrix species in a weight ratio of about 5:1 to about 1:5; and
allowing the dicot plant seed to germinate;
wherein either:
(A) grain yield resulting from the treated dicot plant seed other than rice is at least 2.0% greater than grain yield resulting from the same dicot plant seed that is not exposed to the composition when grown under greenhouse conditions; or
(B) nutrient uptake resulting from the treated dicot plant seed is at least 2% greater than nutrient uptake resulting from the same dicot plant seed that is not exposed to the composition when grown under the greenhouse conditions; or
(C) nitrogen uptake resulting from the treated dicot plant seed is at least 3% greater than nitrogen uptake resulting from the same dicot plant seed that is not exposed to the composition when grown under the greenhouse conditions; or
(D) biomass resulting from the treated dicot plant seed is at least 2% greater than biomass resulting from the same dicot plant seed that is not exposed to the composition when grown under the greenhouse conditions; or
(E) lateral root growth, lateral root hair growth, vertical root growth or root branching resulting from the treated dicot plant seed exhibits increased branching as compared to lateral root growth, lateral root hair growth, vertical root growth or root branching resulting from the same dicot plant seed that is not exposed to the composition when grown under the greenhouse conditions;
wherein the greenhouse conditions are as follows:
(a) seeds are planted in Captina silt loam soil with application of 40 lb/acre phosphate fertilizer and 20 lb/acre potassium fertilizer with 3 plants per pot for hybrid plant and 6 plants per pot for pureline plant;
(b) temperature maintained at 25-28° C., 50-70% relative humidity and natural light supplemented with fluorescent light at 35 μmol m⁻² s⁻² photosynthetically active radiation to provide a 14 h photoperiod; and
(c) plant grown to 50% heading then entire above-ground biomass is collected and dried, and total weight and nitrogen concentration can be measured.

64. (canceled)

65. The treated dicot plant seed of claim 63, wherein yield resulting from the treated dicot plant seed is at least 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, or 25.0% greater than yield resulting from the same dicot plant seed that is not exposed to the composition when grown under the greenhouse conditions.

66. (canceled)

67. The treated dicot plant seed of claim 63, wherein nutrient uptake resulting from the treated dicot plant seed is at least 2, 3, 4, 5, 6, 7, 8, 9, or 10% greater than nutrient uptake resulting from the same dicot plant seed that is not exposed to the composition when grown under the greenhouse conditions.

68. (canceled)

69. The treated dicot plant seed of claim 63, wherein nitrogen uptake resulting from the treated dicot plant seed is at least 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65 or 70% greater than nitrogen uptake resulting from the same dicot plant seed that is not exposed to the composition when grown under the greenhouse conditions.

70. (canceled)

71. The treated dicot plant seed of claim 63, wherein biomass resulting from the treated dicot plant seed is at least 2, 3, 4, 5, 6, 7, 8, 9, 10, 11, 12, 13, 14, 15, 16, 17, 18, 19, 20, 21, 22, 23, 24, or 25% greater than biomass resulting from the same dicot plant seed that is not exposed to the composition when grown under the greenhouse conditions.

72-111. (canceled)


SEQUENCE LISTING (4 sequences)

SEQ ID NO: 1 - DNA, 431 bp, Aulosira
  misc_feature (431)..(434): Consensus
cgaaagcctgacggagcaataccgcgtgagggaggaaggctcttgggtcgtaaacctcttttctcagggaagaacaaaatgacggtacctgaggaataagcatcggctaactccgtgccagcagccgcggtaatacggaggatgcaagcgttatccggaatgattgggcgtaagcgtccgcaggtggctatgtaagtctgctgttaaagagtgaggctcaacctcataaaagcagtggaaactacatggctagagtgcgttcggggcagagggaattcctggtgtagcggtgaaatgcgtagagatcaggaagaacaccggtggcgaaagcgctctgctaggccgcaactgacactgagggacgaaagctaggggagcgaatgggattagataccccagtagtcctgagactccaaggcacacaggggata

SEQ ID NO: 2 - DNA, 401 bp, Anabaena
  misc_feature (75)..(77): consensus
cgaaagcctgacggagcaataccgcgtgagggaggaaggctcttgggtcgtaaacctcttttctcagggaagaaaaatgacggtacctgaggaataagcatcggctaactccgtgccagcagccgcggtaatacggaggatgcaagcgttatccggaatgattgggcgtaagcgtccgcaggtggccatgtaagtctgctgttaaagagtcatgcttaacatgataaaagcagtggaaactacagagctagagtacgttcggggcagagggaattcctggtgtagcggtgaaatgcgtagatatcaggaagaacaccggtggcgaaagcgctctgctaggccgtaactgacactgagggacgaaagctaggggagcgaatgggattagataccccagtagt

SEQ ID NO: 3 - DNA, 378 bp, Nostoc
cgaaagcctgacggagcaataccgcgtgagggaggaaggctcttgggttgtaaacctcttttctcagggaataaaatgaaggtacctgaggaatcagcatcggctaactccgtgccagcagccgcggtaatacggaggatgcaagcgttatccggaatgattgggcgtaagcgtccgcaggtggcgatgtaagtctgctgttaaagagtgaggcttaaacctcataaagcagtggaaactacatcgctagagtgcgttcggggcagagggaattcctggtgtagcggtgaaatgcgtagagatcaggaagaacaccggtggcgaaggcgctctgctaggccgcaactgacactgagggacgaaagctaggggagcgaa

SEQ ID NO: 4 - DNA, 404 bp, Tolypothrix
cgaaagcctgacggagcaataccgcgtgagggaggaaggctcttgggttgtaaacctcttttctcagggaagaattaaatgacggtacctgaggaataagcatcggctaactccgtgccagcagccgcggtaatacggaggatgcaagcgttatccggaatgattgggcgtaaagcgtccgcaggtggctatgtaagtctgctgttaaagaatctggctcaaccagataaaggcagtggaaactacatggctagagtgcgttcggggcagaggggaatttcctggtgtagcggtgaaatgcgtagagatcaggagaacaccggtgcgaaagcgctctgctaggccgcaactgacactgagggacgaaagctaggggagcgaatgggattagataccccagtagt